(19) United States
(12) Patent Application Publication
(10) Pub. No.: US 2012/0103338 A1
(43) Pub. Date: May 3, 2012

(54) Breathing Device

(76) Inventor: Yi-Chang Chan, Xinzhuang City (TW)

(21) Appl. No.: 12/938,909

(22) Filed: Nov. 3, 2010

(51) Int. Cl. A61M 16/20

(52) U.S. Cl. 128/205.24

(57) ABSTRACT

The present invention discloses a breathing device comprising: a main body comprising at least one exhausting port, at least one one-way valve plate, an inlet port, and a stand-connecting part; a air pipe being connected with the inlet port and comprising a check valve disposed on the inner part of the air pipe, wherein the check valve can prevent the air in the air pipe from flowing out of the main body through the air pipe; a plastic stand, wherein one end of the plastic stand is connected with the stand-connecting part of the main body; and a supporting part comprising a first surface and a second surface, wherein the first surface is connected with another end of the plastic stand, and the second surface being provided with at least one cushion element for increasing the comfortability when use.

### BACKGROUND OF THE INVENTION

1. Field of the Invention

The present invention relates to a breathing device, and more particularly to an improved breathing device which makes gas enter the breathing device only from an inlet port and flow out of the breathing device only from an exhausting port.

2. Description of the Prior Art

Obstructive sleep apnea syndrome is a syndrome of a respiratory tract obstruction caused by the collapse of the respiratory tract while sleeping. For treating the syndrome, a continuous positive airway pressure (CPAR) is employed, wherein the CPAR is an air supply machine can be adjustable for the air pressure, and the CPAR can pump the gas with a specific pressure into a breathing mask through an air pipe. The CPAR can help a patient for maintaining the smooth of the respiratory tract and the respiration when sleep, and avoiding the happening of the obstructive sleep apnea syndrome. The CPAR also can improve the problem of snoring.

A conventional breathing mask has the following shortcomings:
1. Vent holes disposed in the breathing mask can discharge the gas breathed out by a user, however, the vent holes may permit the outside gas flow into the breathing mask, so as to decrease the pressure in the breathing mask and reduce the smooth of the respiratory tract.
2. The breathing mask without vent holes should make the gas accumulating in the breathing mask, so as to cause the breathing of the user unwell or a backflow of the gas breathed out by the user, and then the supply of the barotropic gas is affected.
3. A supporting part of the conventional breathing mask is used to contact with the forehead of the user for enhancing the fixation of the breathing mask, however, the supporting part of the conventional breathing mask cannot be bent for various angles, so that supporting part is incapable of contacting with the proper position of the forehead according to the shape of a face.

In view of this, it is necessary to provide an improved breathing device, which can solve the above shortcomings of the conventional breathing mask.

### SUMMARY OF THE INVENTION

In view of the above shortcomings of the prior art, the inventor of the present invention resorted to past experience, imagination, and creativity, performed experiments and researches repeatedly, and eventually devised the present invention, a breathing device.

The major objective of the present invention is to provide the breathing device, which is provided with a one-way valve plate for solving the problem of decreasing the pressure in the breathing mask caused by that the external gas flow into the breathing mask through a vent hole.

Another objective of the present invention is to provide the breathing device, which is provided with a check valve for avoiding that the supply of the barotropic gas affected by a backflow of the gas breathed out by a user.

Further objective of the present invention is to provide the breathing device, which is provided with a flexible stand for assisting a supporting part to contact with a proper position of a forehead according to the shape of a face, so as to increase the stability of the breathing mask fixed on the face.

According to the above objective, the present invention provides a breathing device comprising: a main body comprising: at least one exhausting port for discharging gas from the inner part of the main body to outside; at least one one-way valve plate being disposed on the exhausting port for discharging the gas from the inner part of the main body to outside, and preventing the gas from entering into the main body; an inlet port being the entrance for the gas; a stand-connecting part being disposed on the main body and above the exhausting port; and a belt-connecting part for connecting with a belt which assist the main body to fix on a head; a air pipe being connected with the inlet port and used as a channel for the gas entering the main body, wherein the inner part of the air pipe is provided with a check valve for preventing the gas from discharging from the main body through the air pipe; a plastic stand having one end connected with the stand-connecting part of the main body, and the plastic stand being bent with various angles and for fixing position; and a supporting part for contacting with the head and comprising a first surface and a second surface, wherein the first surface is connected with another end of the plastic stand, and the second surface being provided with at least one cushion element for increasing the comfortability when the supporting part contact with the head.

### BRIEF DESCRIPTION OF THE DRAWINGS

For a complete understanding of the aspects, structures and techniques of the invention, reference should be made to the following detailed description and accompanying drawings wherein:

FIG. 1 is an exploded view of a breathing device according to a preferred embodiment of the present invention;

FIG. 2 is a perspective view of the breathing device from a first angle according to the preferred embodiment of the present invention;

FIG. 3 is a perspective view of the breathing device from a second angle according to the preferred embodiment of the present invention; and

FIG. 4 is an exploded view of an air hole of the breathing device according to the preferred embodiment of the present invention.

### DETAILED DESCRIPTION OF THE PREFERRED EMBODIMENTS

To achieve the foregoing objectives and effects, the inventors improve the conventional breathing mask, thus achieving a breathing device of the present invention. Hereinafter, the breathing device according to a preferred embodiment of the present invention will be described in detail for illustrating the structural features and principles of the present invention.

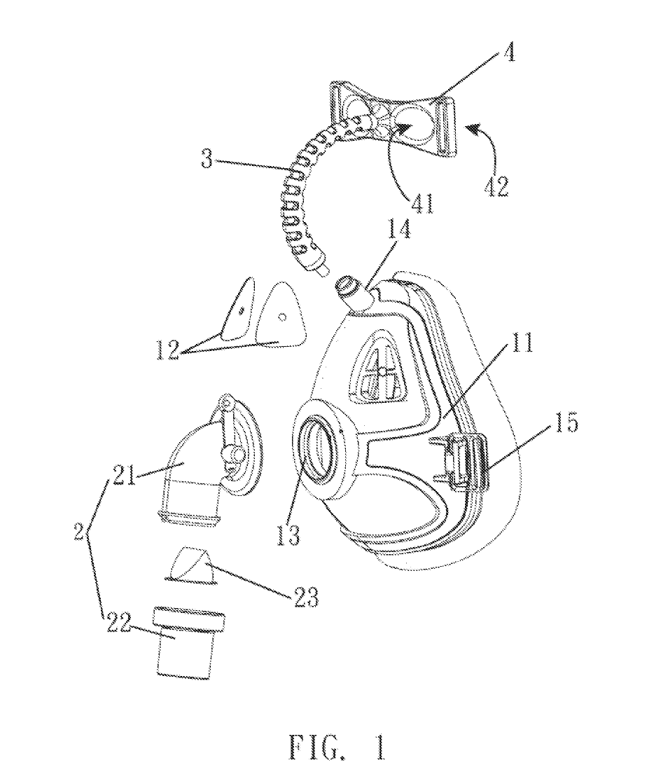

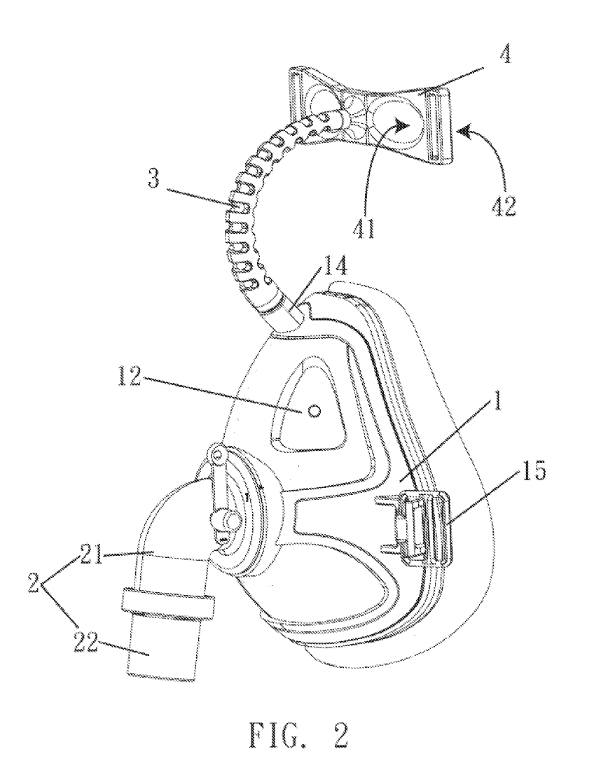

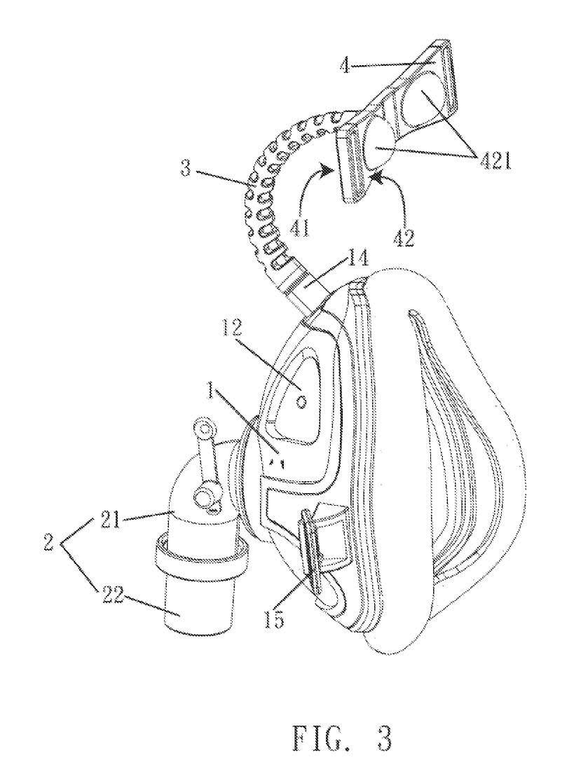

Referring to FIG. 1, FIG. 2 and FIG. 3, FIG. 1 is an exploded view of the breathing device according to the preferred embodiment of the present invention, FIG. 2 is a perspective view of the breathing device from a first angle according to the preferred embodiment of the present invention, and FIG. 3 is a perspective view of the breathing device from a second angle according to the preferred embodiment of the present invention. The breathing device includes a main body 1, a air pipe 2, a plastic stand 3, and a supporting part 4.

The main body 1 includes: a set of exhausting port 11 for discharging gas from the inner part of the main body 1 to outside; a set of one one-way valve plates 12 being disposed on the exhausting port 11 for discharging the gas from the inner part of the main body 1 to outside, and preventing the gas from entering into the main body 1; an inlet port 13 being the entrance for the gas flowing into the main body 1; a stand-connecting part 14 being disposed on the main body 1 and above the exhausting port 11; and a belt-connecting part 15 for connecting with a belt (not shown in the figures) which assist the main body 1 to fix on a head.

The air pipe 2 is connected with the inlet port 13 and used as a channel for the external gas entering the main body. The inner part of the air pipe 2 is provided with a check valve 23 for preventing the inner gas from discharging from the main body 1 through the air pipe 2. The air pipe 2 can be disassembled into a first pipe element 21 and a second pipe element 22, and the check valve 23 is disposed on the joint between the first pipe element 22 and the second pipe element 23 for easy replacement.

The plastic stand 3 has one end connected with the stand-connecting part 14 of the main body 1, and the plastic stand 3 can be bent with various angles and used for fixing position.

The supporting part 4 is configured to contact with the head. The supporting part 4 includes a first surface 41 and a second surface 42, wherein the first surface 41 is connected with another end of the plastic stand 3, and the second surface 42 is provided with a set of cushion elements 421 for increasing the comfortability when the supporting part 4 contact with the head. The set of cushion elements 421 is a set of gasbags.

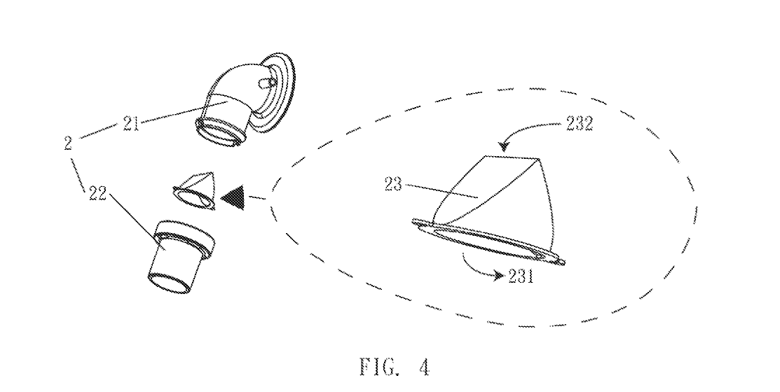

Referring to FIG. 4, which is an exploded view of an air hole of the breathing device according to the preferred embodiment of the present invention. In the preferred embodiment of the present invention, the check valve 23 is a pyramid, and includes an inlet end 231 and an exhaust end 232. The area of the inlet end 231 is greater than the area of the exhaust end 232, so that the intake pressure in the inlet end 231 is greater than the discharge pressure in the exhaust end 232 and the backflow of the gas in the main body 1 through the gas pipe 2 can be prevented.

By the detailed description of the overall structure and technical content of the present invention, the following advantages of the present invention can be derived:

The one-way valve plate can solve the problem of decreasing the pressure in the breathing mask caused by that the external gas flow into the breathing mask through a vent hole.

The check valve can maintain the supply of the barotropic gas and avoid the backflow of the gas breathed out by a user.

The flexible stand can be bent for various angles for assisting a supporting part to contact with a proper position of a forehead according to the shape of a face, so as to increase the stability and the comfortability of the breathing mask fixed on the face.

It should be understood that the embodiments of the present invention described herein are merely illustrative of the technical concepts and features of the present invention and are not meant to limit the scope of the invention. Those skilled in the art, after reading the present disclosure, will know how to practice the invention. Various variations or modifications can be made without departing from the spirit of the invention. All such equivalent variations and modifications are intended to be included within the scope of the invention.

As a result of continued thinking about the invention and modifications, the inventors finally work out the designs of the present invention that has many advantages as described above. The present invention meets the requirements for an invention patent, and the application for a patent is duly filed accordingly. It is expected that the invention could be examined at an early date and granted so as to protect the rights of the inventors.

What is claimed is:

1. A breathing device comprising:
a main body comprising:
at least one exhausting port for discharging gas from the inner part of the main body to outside;
at least one one-way valve plate being disposed on the exhausting port for discharging the gas from the inner part of the main body to outside, and preventing the gas from entering into the main body;
an inlet port being the entrance for the gas;
a stand-connecting part being disposed on the main body and above the exhausting port; and
a belt-connecting part for connecting with a belt which assist the main body to fix on a head;
a air pipe being connected with the inlet port and used as a channel for the gas entering the main body, wherein the inner part of the air pipe is provided with a check valve for preventing the gas from discharging from the main body through the air pipe;
a plastic stand having one end connected with the stand-connecting part of the main body, and the plastic stand being bent with various angles and for fixing position; and
a supporting part for contacting with the head and comprising a first surface and a second surface, wherein the first surface is connected with another end of the plastic stand, and the second surface being provided with at least one cushion element for increasing the comfortability when the supporting part contact with the head.

2. The breathing device according to claim 1, wherein the check valve is a pyramid and comprises an inlet end and an exhaust end, and wherein the area of the inlet end is greater than the area of the exhaust end, so that the intake pressure in the inlet end is greater than the discharge pressure in the exhaust end and the backflow of the gas in the breathing device can be prevented.

3. The breathing device according to claim 1, wherein the air pipe comprises a first pipe element and a second pipe element, and the check valve being disposed on the joint between the first pipe element and the second pipe element for easy replacement.

4. The breathing device according to claim 1, wherein the cushion element can be a gasbag.
